(19) United States
(12) Patent Application Publication
(10) Pub. No.: US 2026/0076613 A1
(43) Pub. Date: Mar. 19, 2026

(54) METHOD FOR MONITORING VITAL SIGNS OF POST-OPERATIVE ORGAN TRANSPLANT PATIENTS

(71) Applicant: The First Affiliated Hospital of Guangzhou Medical University (The center of Guangzhou Respiratory), Guangzhou City (CN)

(72) Inventors: Danxia HUANG, Guangzhou City (CN); Minting KUANG, Guangzhou City (CN); Run LI, Guangzhou City (CN)

(73) Assignee: The First Affiliated Hospital of Guangzhou Medical University (The center of Guangzhou Respiratory), Guangzhou City (CN)

(21) Appl. No.: 19/184,091

(22) Filed: Apr. 21, 2025

(30) Foreign Application Priority Data
Jun. 7, 2024 (CN) 2024107326956

(51) Int. Cl. A61B 5/00; A61B 5/117

(52) U.S. Cl. CPC A61B 5/413; A61B 5/117; A61B 5/6804; A61B 5/725; A61B 5/7264

(57) ABSTRACT

Provided herein is a method for monitoring vital signs of a post-operative organ transplant patient, comprising collecting patient information, selecting a wearable device, and monitoring real time vital signs data using the wearable device, comprehensively harmonizing the acquired real-time vital signs data, and storing the comprehensively harmonized vital signs data; periodically retrieving the vital signs data of the patient, verifying and correcting the vital signs data of the patient using a data verification and correction algorithm, and processing and analyzing the verified and corrected vital signs data of the patient to generate a health status report of the patient; and encrypting the health status report of the patient using a health security encryption technology, and introducing a fast and secure transmission method.

### CROSS-REFERENCE TO RELATED APPLICATIONS

This disclosure claims priority under 35 U.S.C. § 119(a) to Chinese Patent No. 2024107326956, filed on Jun. 7, 2024, which is hereby incorporated by reference herein in its entirety.

### TECHNICAL FIELD

The present disclosure relates to a technical field of vital sign monitoring, in particular to a method for monitoring vital signs of a post-operative organ transplant patient.

### BACKGROUND ART

Monitoring vital signs in a patient after organ transplantation is of critical importance, as it enables a timely detection of changes in the patient's physical condition and allows medical professionals to take prompt and appropriate therapeutic interventions, thereby increasing the patient's chances of survival. At present, the method for monitoring vital signs of post-operative organ transplant patients mainly depends on hospital-based monitoring devices, such as electrocardiogramachines, sphygmomanometers, and pulse oxygen saturation monitor. These devices are capable of monitoring vital signs including such as heart rate, blood pressure, and blood oxygen saturation in real time.

However, these traditional monitoring devices have some limitations. First of all, these devices typically require a patient to remain hospitalized for monitoring, which not only increases a financial burden on the patient but also restricts their ability to engage in daily activities. Secondly, using such devices usually requires trained medical personnel, thereby increasing hospital staffing costs. In addition, although these devices offer real-time monitoring, they often lack the capability for long-term data storage and analysis, which limits a physician's ability to fully assess a patient's overall condition.

Various methods for vital sign monitoring have been proposed. CN116509365A, Cao Lin et al. discloses a method and device for monitoring vital signs. This method mainly includes the following steps: acquiring echo data; performing 3D-FFT processing on the echo data to determine whether a target is present; upon detecting the presence of the target, performing a target detection to obtain micro-Doppler signals corresponding to vital signs; extracting phase information from the micro-Doppler signals; filtering the phase information using a first bandpass filter and a second bandpass filter to obtain a first phase data and a second phase data; performing a frequency tracking on the first phase data and the second phase data using a M-Rife interpolation algorithm to obtain a first frequency value and a second frequency value; wherein the first frequency value represents a first vital sign, and the second frequency value represents a second vital sign.

### SUMMARY

The present disclosure provides a method for monitoring vital signs of post-operative organ transplant patient, which enables timely and accurate monitoring during vital sign measurement.

Various embodiments provides a method for monitoring vital signs of a post-operative organ transplant patient, comprising:
Step S1. collecting patient information, selecting a wearable device for the patient, and monitoring real time vital signs data of the patient using the wearable device, comprehensively harmonizing the acquired real time vital signs data, and storing the comprehensively harmonized vital signs data;
Step S2. retrieving the vital signs data of the patient, verifying and correcting the vital signs data of the patient using a data verification and correction algorithm, and processing and analyzing the verified and corrected vital signs data of the patient to generate a health status report of the patient; and
Step S3. encrypting the health status report of the patient using a health security encryption technology, and introducing a fast and secure transmission method.

According to one or more embodiments, the step S1 comprises:
collecting the patient information from a hospital medical record system;
selecting the wearable device for the patient based on the patient information by medical personnel;
capturing the real time vital signs data of the patient using the wearable device; and
comprehensively harmonizing the acquired real-time vital signs data.

According to one or more embodiments, comprehensively harmonizing the acquired real time vital signs data is performed by introducing:
a data cleansing and purification algorithm for cleansing and purifying the acquired real-time vital signs data,
a data denoising algorithm for reducing noise in the acquired real-time vital signs, and
a data normalization and balancing algorithm for balancing the acquired real-time vital signs data.

According to one or more embodiments, the step S1 further comprises:
applying a deep learning-based data compression algorithm to the harmonized vital signs data, automatically selecting an optimal compression ratio based on the characteristics of the vital signs data to compress the vital signs data;
encoding, decoding, and parsing the compressed data to form a data packet, thereby obtaining a packaged and compressed patient vital signs dataset;
transmitting and storing the packaged and compressed patient vital signs data; and
introducing a blockchain-based data transmission protocol to transmit the packaged and compressed patient vital signs data to a database for storage.

According to one or more embodiments, the step S2 comprises:
retrieving the vital signs data of the patient from a database;
verifying and correcting the vital signs data of the patient;
extracting features from the verified and corrected vital signs data of the patient to obtain a comprehensive feature vector representing the vital signs of the patient; and
analyzing the comprehensive feature vector representing the vital signs of the patient to generate a health status report of the patient.

According to one or more embodiments, the step S2 further comprises:
extracting statistical features from the verified and corrected vital signs data of the patient using a statistical analysis method and calculating statistical characteristics of each vital sign;
transforming the verified and corrected vital signs data of the patient from a time domain to a frequency-domain using a Fourier transform to obtain frequency-domain feature values of the vital signs data; and
introducing a mutual information-based frequency-domain feature selection algorithm to obtain filtered frequency-domain feature values.

According to one or more embodiments, the step S2 further comprises:

introducing a comprehensive feature fusion algorithm; wherein the comprehensive feature fusion algorithm fuses the statistical features of the vital signs of the patient and the filtered frequency-domain feature to form a comprehensive feature vector.

According to one or more embodiments, the step S2 further comprises:
standardizing the comprehensive feature vector of the vital signs of the patient during data analysis;
selecting features most relevant to a health status of the patient using a principal component analysis algorithm;
establishing a health status prediction model under training of a machine learning model;
obtaining a predicted health status result of the patient using the health status prediction model; and
introducing a regularized support vector machine algorithm during training of the health status prediction model.

According to one or more embodiments, the step S3 comprises:
encrypting the health status report of the patient using a health security encryption technology; and
optimizing the health security encryption technology.

In the present disclosure, the completeness, accuracy, and stability of vital sign data of a patient is ensured by employing a data cleansing and purification algorithm, a data denoising algorithm, and a data normalization and balancing algorithm. These high-quality data serve as a basis for subsequent analysis. Additionally, a deep learning-based data compression algorithm is utilized to automatically select an optimal compression ratio based on the characteristics of the data, thereby maintaining the quality of the data after compression and reducing storage requirements. The integrity and security of the data during transmission are ensured through a blockchain-based data transmission protocol, which prevent tampering of the data in transit. Furthermore, a distributed storage system is employed, wherein data is encrypted and decrypted and stored through fragmentation and recombination using established techniques, thereby ensuring a secure data management.

Statistical features are extracted using a statistical analysis method, and frequency-domain features are extracted using a Fourier transform. A mutual information-based frequency-domain feature selection algorithm is then introduced to identify key frequency components associated with a patient's health status, thereby improving the accuracy and efficiency of data analysis. Statistical features and frequency-domain features are then fused into a comprehensive feature vector using a feature fusion algorithm. A machine learning algorithm is employed to establish a health status prediction model, generate a prediction result of the patient, and produce a health status report of the patient, which can be accessed conveniently by both physicians and patients at any time and from any location.

A health data security encryption technique based on the AES (Advanced Encryption Standard) algorithm is employed to encrypt the health status report of a patient, ensuring that only authorized medical personnel and the patient have access to the data. This protects patient privacy and prevents data leakage or unauthorized access. Additionally, a fast and secure transmission method is introduced, using data compression techniques to enable efficient transmission, reduce transmission time, and increase transmission speed, allowing physicians and patients to receive a health report more quickly.

The technical solution of the present application enables timely and accurate monitoring during vital sign measurement. The completeness, accuracy, and stability of vital sign data of a patient is ensured by employing a data cleansing and purification algorithm, a data denoising algorithm, and a data normalization and balancing algorithm. These high-quality data serve as a basis for subsequent analysis. A deep learning-based data compression algorithm is utilized to automatically select an optimal compression ratio based on the characteristics of the data, thereby maintaining the quality of the data after compression and reducing storage requirements. The integrity and security of the data during transmission are ensured through a blockchain-based data transmission protocol, which prevent tampering of the data in transit. A distributed storage system is employed, wherein data is encrypted and decrypted using established cryptographic techniques and stored through fragmentation and recombination, ensuring a secure data management. Statistical features are extracted using a statistical analysis method, and frequency-domain features are extracted using a Fourier transform. A mutual information-based frequency-domain feature selection algorithm is then introduced to identify key frequency components associated with a patient's health status, thereby improving the accuracy and efficiency of data analysis. Statistical features and frequency-domain features are then fused into a comprehensive feature vector using a feature fusion algorithm. A machine learning algorithm is employed to establish a health status prediction model, generate a prediction result of the patient, and produce a health status report of the patient, which can be accessed conveniently by both physicians and patients at any time and from any location. A health data security encryption technique based on the AES (Advanced Encryption Standard) algorithm is employed to encrypt the health status report of a patient, thereby ensuring that only authorized medical personnel and the patient have access to the data. This protects patient privacy and prevents data leakage or unauthorized access. Additionally, a fast and secure transmission method is introduced, using data compression techniques to enable efficient transmission, reduce transmission time, and increase transmission speed, allowing physicians and patients to receive a health report more quickly.

### BRIEF DESCRIPTION OF THE DRAWINGS

FIG. 1 is a flow chart of a method for monitoring vital signs of a post-operative organ transplant patient according to various embodiments.

### DETAILED DESCRIPTION

The present disclosure provides a method for monitoring vital signs of a post-operative organ transplant patient, and various embodiments of the method are at least based on one or more of the following concepts:

Firstly, collecting basic information of a patient, selecting an appropriate wearable device for the patient, monitoring vital signs data of the patient using the wearable device, comprehensively harmonizing the acquired vital signs data, and storing the comprehensively harmonized vital signs data, so as to provide a data basis for subsequent verification and correction. Secondly, periodically retrieving the vital signs data of the patient, verifying and correcting the vital signs data of the patient using a data verification and correction algorithm, and processing and analyzing the verified and corrected vital signs data of the patient to generate a health status report of the patient. Finally, encrypting the health status report of the patient using a health security encryption technology so that only physicians and patients can view the data, while a fast and secure data transmission between the database and physicians and patients is realized by introducing a fast and secure transmission method. The completeness, accuracy, and stability of the vital sign data of the patient is ensured by employing a data cleansing and purification algorithm, a data denoising algorithm, and a data normalization and balancing algorithm. These high-quality data serve as a basis for subsequent analysis. A deep learning-based data compression algorithm is utilized to automatically select an optimal compression ratio based on the characteristics of the data, thereby maintaining the quality of the data after compression and reducing storage requirements. The integrity and security of the data during transmission are ensured through a blockchain-based data transmission protocol, which prevent tampering of the data in transit. A distributed storage system is employed, wherein data is encrypted and decrypted using established cryptographic techniques and stored through fragmentation and recombination, thereby ensuring a secure data management. Statistical features are extracted using a statistical analysis method, and frequency-domain features are extracted using a Fourier transform. A mutual information-based frequency-domain feature selection algorithm is then introduced to identify key frequency components associated with a patient's health status, thereby improving the accuracy and efficiency of data analysis. Statistical features and frequency-domain features are then fused into a comprehensive feature vector using a feature fusion algorithm. A machine learning algorithm is employed to establish a health status prediction model, generate a prediction result of the patient, and produce a health status report of the patient, which can be accessed conveniently by both physicians and patients at any time and from any location. A health data security encryption technique based on the AES (Advanced Encryption Standard) algorithm is employed to encrypt the health status report of a patient, ensuring that only authorized medical personnel and the patient have access to the data. This protects patient privacy and prevents data leakage or unauthorized access. Additionally, a fast and secure transmission method is introduced, using data compression techniques to enable efficient transmission, reduce transmission time, and increase transmission speed, allowing physicians and patients to receive a health report more quickly.

To better understand the principles of the present disclosure, a detailed description will be provided below in conjunction with the accompanying drawings and various embodiments.

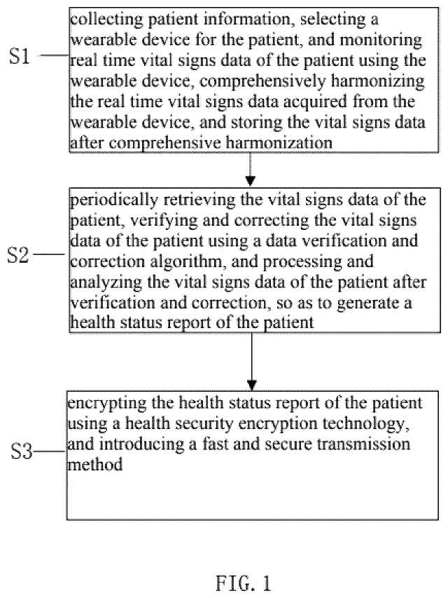

Referring to an embodiment shown in FIG. 1, the method for monitoring vital signs of the post-operative organ transplant patient includes at least one or more of the following exemplary steps.

Step S1: collecting basic information of a patient, selecting a wearable device for the patient, and monitoring real time vital signs data of the patient using the wearable device, comprehensively harmonizing the acquired real-time vital signs data, and storing the comprehensively harmonized vital signs data.

According to some examples, in the step S1, the basic information of the patient is collected from a hospital medical record system, the basic information of the patient including name, age, gender and medical history. The basic information is considered as a basis of device monitoring and data analysis. The appropriate wearable device is selected based on the patient information (including health status and family status) by medical personnel. In some examples, the wearable device is integrated with a plurality of sensors, such as a heart rate sensor, a blood pressure sensor and a blood oxygen saturation sensor.

When the patient wears the wearable device, the medical personnel turns on the wearable device and carries out initialization settings, such as time and date. According to the basic information of the patient, parameters of the wearable device are adjusted and calibrated to ensure an accuracy of the monitoring data. The vital sign data of the patient in real time is captured by the wearable device. In some examples, the vital sign data include heart rate, blood pressure and oxygen saturation.

According to some embodiments, comprehensively harmonizing the acquired real-time vital signs data is carried out through the following exemplary process.

Firstly, for real-time vital sign data of the patient, a data cleansing and purification algorithm is introduced. This algorithm first identifies and removes anomalous data that do not conform to physiological norms, then fills in missing data points using an interpolation method and finally performs a deduplication to ensure the integrity and consistency of the data, thereby producing cleansed and purified vital sign data. Next, for the cleansed and purified vital sign data, a data denoising algorithm is introduced. This algorithm first decomposes the data using wavelet transform, then removes noise by a thresholding method, and finally reconstructs the data using an inverse wavelet transform to ensure the smoothness and accuracy of the data, thereby producing denoised vital sign data. Subsequently, for the denoised data, a data normalization and balancing algorithm is introduced. This algorithm first calculates a maximum value and a minimum value of the data, then maps the data to the [0, 1] range using a linear transformation, and finally standardizes the data to ensure comparability and stability, thereby producing normalized and balanced vital sign data.

Through the above steps, a comprehensive harmonization of vital sign data of the patient is achieved, including cleansing and purification, denoising, and normalization and balancing, thereby providing high-quality data for subsequent data analysis.

For the comprehensively harmonized vital sign data of the patient, a deep learning-based data compression algorithm is introduced. This algorithm is capable of automatically selecting an optimal compression ratio based on the characteristics of the data to ensure the quality of the data after compression. Leveraging the strong feature-learning capability of deep neural networks, this algorithm autonomously learns data characteristics and determines an optimal compression ratio accordingly. The comprehensively harmonized vital sign data is input into the deep learning model, which automatically analyzes the features of the data and selects an optimal compression ratio for compression. The implementation process adopts optimization algorithms such as forward propagation and backward propagation of multi-layer neural network and gradient descent. Furthermore, the compressed data is encoded, decoded, and parsed using packaging techniques to generate a data packet, thereby obtaining a packaged and compressed vital sign data of the patient.

According to some embodiments, the packaged and compressed vital sign data of the patient are transmitted and stored to serve as a basis for further analysis. During data transmission, a blockchain-based data transmission protocol is introduced, which ensures the security and integrity of the data throughout the transmission process, and prevents the data from being tampered throughout the transmission process by using the non-tampering of blockchain. The packaged and compressed vital sign data of the patient are transmitted to a database for storage via the blockchain protocol. The implementation process involves data encryption and decryption, as well as blockchain generation and verification. Upon receiving the data, the database will first verify the data using the blockchain verification and data decryption algorithm to ensure the integrity and security of the data. During storage, a distributed storage system is employed. In this system, the data is subjected to fragmentation and recombination using established techniques, along with encryption and decryption algorithms, to further ensure secure and reliable storage of the vital sign data of the patient.

According to the present disclosure, the completeness, accuracy, and stability of vital sign data of a patient is ensured by employing the data cleansing and purification algorithm, the data denoising algorithm, and the data normalization and balancing algorithm. These high-quality data serve as a basis for subsequent analysis. A deep learning-based data compression algorithm is utilized to automatically select an optimal compression ratio based on the characteristics of the data, thereby maintaining the quality of the data after compression and reducing storage requirements. The integrity and security of the data during transmission are ensured through a blockchain-based data transmission protocol, which prevent tampering of the data in transit. The distributed storage system is employed, wherein the data is encrypted and decrypted, and stored through fragmentation and recombination using established techniques, ensuring a secure data management.

Step S2: periodically retrieving the vital signs data of the patient, verifying and correcting the vital signs data of the patient using a data verification and correction algorithm, and processing and analyzing the verified and corrected vital signs data of the patient to generate a health status report of the patient.

In some examples, the step S2 includes a substep S21: performing data verification and fusion correction processing.

In the substep S21, periodically retrieving the vital signs data of the patient from a database, and performing a verification process on the retrieved vital sign data to ensure the integrity and validity of the data. In some examples, the verification process includes the following:

Range locking of data: verifying whether a data value fall within a reasonable physiological limit. For example, a normal heart rate range is 60-100 beats per minute. If the data falls outside this range, it is marked as abnormal data.

Data type validation: checking whether a data type conforms to an expected format. For instance, a blood pressure value should be an integer or a floating-point number. If the format does not meet this requirement, the data is marked as abnormal data.

Consistency review of data: examining a logical consistency between different data points. For example, a systolic blood pressure should be greater than a diastolic blood pressure. If this relationship is not maintained, the data is marked as abnormal data.

Integrity verification of data: ensuring the completeness of each data. For example, a complete vital sign record for a patient should include a heart rate, a blood pressure, and a blood oxygen saturation. If any of these parameters are missing, the data is marked as incomplete and abnormal data.

In some embodiments, for the vital sign data of the patient that fail the verification process, a data fusion correction algorithm is introduced to correct the invalid data, thereby ensuring the accuracy and stability of the data. The fusion correction method includes the following exemplary steps.

Firstly, an interpolation is performed on data that fail the verification, wherein missing values are filled using the average of adjacent data; a fluctuation smoothing is applied to the data, and abnormal values are smoothed using the average of neighboring data points; a consistency correction is performed on inconsistent data, in which inconsistent values are adjusted based on the average of surrounding data to ensure coherence; a trend analysis is conducted on the unverified data using a time-series analysis method to evaluate a trend of changes in the vital signs of the patient, and any abnormal trends identified are corrected accordingly; and an anomaly detection is carried out on the unverified data to identify and mark abnormal data. The implementation process is as follows:

Q1=a first quartile, Q3=a third quartile, and IQR=Q3−Q1; where IQR is a quartile spacing; and If xᵢ<Q1−1.5×IQR or xᵢ>Q3+1.5×IQR, xᵢ is marked as an abnormal value.

In some embodiments, the data is subjected to normalization and standardization processing. In the normalization process, the data is mapped to a range between 0 and 1 to facilitate subsequent analysis and processing. In the standardization process, the data is transformed into a distribution with a mean of 0 and a standard deviation of 1, further supporting subsequent data analysis and processing.

In some embodiments, the step S2 includes a substep S22: processing and analyzing the verified and corrected data.

In some examples, the substep S22 includes feature extraction.

In the feature extraction, for the verified and corrected data, a statistical analysis method is employed to extract statistical features. Statistical features for each type of vital sign are calculated, which may include the mean, maximum, minimum, and standard deviation;

Fourier transform is used to the verified and corrected data to convert the vital sign signals from the time domain to the frequency-domain, from which the principal frequency components and their corresponding amplitudes are extracted, thereby obtaining frequency-domain features of the vital signs;

In some examples, in order to identify key frequency components from a large number of frequency-domain features that are relevant to the health status, a mutual information-based frequency-domain feature selection algorithm is introduced. This algorithm calculates a mutual information value between each frequency-domain feature and the health status, ranks the features according to their mutual information values, and selects a top N frequency-domain features with the highest mutual information values (where N is empirically determined), thereby obtaining a filtered key frequency-domain feature. The specific implementation is as follows:

Firstly, the mutual information value of each frequency-domain feature and health status is calculated. For each frequency-domain feature Xᵢ and health status Y, the mutual information MI(Xᵢ; Y) is defined as:

$$MI ( {X}_{i} ; Y ) = {\sum }_{x ∈ {X}_{i}} {\sum }_{y ∈ Y} p ( x , y ) log ( \frac{p ( x , y )}{p ( x ) p ( y )} )$$

where p(x, y) is a joint probability density function, and p(x) and p(y) are a marginal probability density function respectively; in order to improve the accuracy, a weight factor wᵢ is introduced to represent the importance of each frequency-domain feature. The calculation formula of the weight factor is as follows:

$${w}_{i} = \frac{1}{1 + {e}^{- α MI ( {X}_{i} ; Y )}}$$

where α is an adjustment parameter used to control a rate of change of the weight factor, and its value can be adjusted according to practical requirements.

In some embodiments, the frequency-domain features are ranked based on their mutual information values, and a top N frequency-domain features with the largest mutual information value are selected. A scoring function Sᵢ is defined to represent the importance of each frequency-domain feature:

$${S}_{i} = {w}_{i} \cdot MI ( {X}_{i} ; Y )$$

All frequency-domain features are then ranked according to a value of the scoring function, and the top N frequency-domain features with the largest scoring function value are selected, where N is empirically determined.

According to some embodiments, in order to better combine statistical features and frequency-domain features to evaluate a health status, a comprehensive feature fusion algorithm is introduced. The comprehensive feature fusion algorithm fuses the statistical features of vital signs and the filtered key frequency-domain features to form a comprehensive feature vector, which is specifically realized as follows:

The statistical features and frequency-domain features are fused to form a comprehensive feature vector V:

$$V = [ {V}_{s} , {V}_{f} ]$$

where Vₛ is a statistical feature vector and V_{f} is a frequency-domain feature vector.

In order to improve the accuracy of fusion, a fusion coefficient β is introduced to adjust the weighting between the statistical features and the frequency-domain features. The calculation formula of the fusion coefficient β is as follows:

$$β = \frac{{\sum }_{i = 1}^{n} {w}_{i} \cdot MI ( {X}_{i} ; Y )}{{\sum }_{i = 1}^{n} MI ( {X}_{i} ; Y )}$$

where n is a total number of frequency-domain features.

The calculation formula of the comprehensive feature vector V is as follows:

$$V = β \cdot {V}_{s} + ( 1 - β ) {V}_{f}$$

As a result, a comprehensive feature vector of the vital signs of the patient is obtained.

In some examples, the substep S22 includes data analysis.

In the data analysis, the comprehensive feature vector of the vital signs is standardized so that it has a mean of 0 and a variance of 1, thereby eliminating the influence of dimensional and scale differences in the data. A principal component analysis algorithm is then used to select the features most related to a health status and obtain a selected key feature vectors. A machine learning algorithm is used to train the data and establish a health status predictive model. The key feature vectors of patients are evaluated using the health status prediction model to obtain a predicted health status result of the patient.

In the process of training a model, in order to avoid over-fitting of the model and ensure that the model is of good generalization ability, a regularization support vector machine algorithm is introduced. This algorithm incorporates the results of mutual information-based feature selection, and by introducing a regularization term, over-fitting is prevented and the generalization capability of the model is ensured.

According to some embodiments, the analysis results of the patient's health status are integrated, which include the patient's vital sign data and the health condition assessment. A data structure is created to store the integrated analysis results, which include the patient's basic information, vital sign data, and health status analysis results. The analysis results are then filled into the data structure according to a predefined format to ensure the completeness and accuracy of the data.

Next, based on the integrated analysis results, a health status report of the patient is generated. The health status report is generated using a pre-designed report template. The health status report can include a title of the report, basic information of a patient, vital sign data and health status analysis results. The integrated analysis results are then filled into the report template to generate a health report of the patient. Finally, the health report is formatted to ensure its readability and visual clarity.

Finally, the health report of the patient is uploaded to a database, which is convenient for physicians and patients to access anytime and anywhere. Access permissions are configured to ensure that only authorized physicians and patients can access the report.

In this disclosure, statistical features are extracted using a statistical analysis method, and frequency-domain features are extracted using a Fourier transform. A mutual information-based frequency-domain feature selection algorithm is then introduced to identify key frequency components associated with a patient's health status, thereby improving the accuracy and efficiency of data analysis. Statistical features and frequency-domain features are then fused into a comprehensive feature vector using a feature fusion algorithm. A machine learning algorithm is employed to establish a health status prediction model, generate a prediction result of the patient, and produce a health status report of the patient, which can be accessed conveniently by both physicians and patients at any time and from any location.

Step S3: encrypting the health status report of the patient using a health security encryption technology, and introducing a fast and secure transmission method.

In order to protect the security of the health status report of the patient, a health security encryption technology is introduced, which adds an additional layer of security and irreversibility based on the existing AES (Advanced Encryption Standard) algorithm. The specific implementation process is as follows:
selecting a 256-bit key K and converting it into a matrix form, wherein the key K is a secret parameter in the encryption process and can only be accessed by authorized physicians and patients;
converting the health status report of the patient into a matrix form, denoted as M, and recording that the health status report M is the patient's medical information, which can include the patient's basic information, medical history and medication usage;
encrypting the M by the AES algorithm to obtain an encrypted matrix C; and
performing an exclusive OR (XOR) operation between the encrypted matrix C and the key K to obtain a final encrypted result E, expressed as:

$$E = AES ( M ) ⊕ K$$

where ⊕ represents the XOR operation.

According to some embodiments, in order to increase the accuracy of the encryption technology, the disclosure performs a series of optimization on the above encryption technology, and the specific formula is expressed as follows:

$$F ( M , K , N , P , H ) = ( AES ( M ) ⊕ K ) \times ( \frac{N}{P + H} )$$

where N is a number of bytes in the health status report of the patient; P is a patient's personal information; H is a health status of patients; F(M, K, N, P, H) is an encryption process of the report representing the patient's health status; and

$$\frac{N}{P + H}$$

is a number of bytes in the health status report of the patient divided by a sum of the patient's personal information and health status.

According to some embodiments, in order to improve the speed of data transmission, the present disclosure introduces a fast and secure transmission method, which utilizes data compression technology to achieve efficient data transfer. The specific implementation process is as follows:

Firstly, based on the encrypted health report of the patient E, data is compressed to obtain compressed data Z; Then the compressed data Z will be transmitted to a destination, where Z will be decompressed to recover E; Finally, an inverse process of the health security encryption technology is applied to decrypt E, thereby obtaining the patient's health status report.

In this disclosure, a health data security encryption technique based on the AES (Advanced Encryption Standard) algorithm is employed to encrypt the health status report of a patient, ensuring that only authorized medical personnel and the patient have access to the data. This protects patient privacy and prevents data leakage or unauthorized access. Additionally, a fast and secure transmission method is introduced, using data compression techniques to enable efficient transmission, reduce transmission time, and increase transmission speed, allowing physicians and patients to receive a health report more quickly.

The steps of the method described in the above embodiments are merely illustrative. The order of some of these steps may be interchanged as appropriate without departing from the scope of the present disclosure.

In this disclosure, the completeness, accuracy, and stability of vital sign data of a patient is ensured by employing a data cleansing and purification algorithm, a data denoising algorithm, and a data normalization and balancing algorithm. These high-quality data serve as a basis for subsequent analysis. Additionally, a deep learning-based data compression algorithm is utilized to automatically select an optimal compression ratio based on the characteristics of the data, thereby maintaining the quality of the data after compression and reducing storage requirements. The integrity and security of the data during transmission are ensured through a blockchain-based data transmission protocol, which prevent tampering of the data in transit. Furthermore, a distributed storage system is employed, wherein data is encrypted and decrypted and stored through fragmentation and recombination using established techniques, ensuring a secure data management.

Statistical features are extracted using a statistical analysis method, and frequency-domain features are extracted using a Fourier transform. A mutual information-based frequency-domain feature selection algorithm is then introduced to identify key frequency components associated with a patient's health status, thereby improving the accuracy and efficiency of data analysis. Statistical features and frequency-domain features are then fused into a comprehensive feature vector using a feature fusion algorithm. A machine learning algorithm is employed to establish a health status prediction model, generate a prediction result of the patient, and produce a health status report of the patient, which can be accessed conveniently by both physicians and patients at any time and from any location.

A health data security encryption technique based on the AES (Advanced Encryption Standard) algorithm is employed to encrypt the health status report of a patient, ensuring that only authorized medical personnel and the patient have access to the data. This protects patient privacy and prevents data leakage or unauthorized access. Additionally, a fast and secure transmission method is introduced, using data compression techniques to enable efficient transmission, reduce transmission time, and increase transmission speed, allowing physicians and patients to receive a health report more quickly.

### Performance Evaluation:

The technical solution of the present application enables timely and accurate monitoring during vital sign measurement. The proposed system or method has undergone a series of performance evaluations. The completeness, accuracy, and stability of vital sign data of a patient is ensured by employing a data cleansing and purification algorithm, a data denoising algorithm, and a data normalization and balancing algorithm. These high-quality data serve as a basis for subsequent analysis. Additionally, a deep learning-based data compression algorithm is utilized to automatically select an optimal compression ratio based on the characteristics of the data, thereby maintaining the quality of the data after compression and reducing storage requirements. The integrity and security of the data during transmission are ensured through a blockchain-based data transmission protocol, which prevent tampering of the data in transit. Furthermore, a distributed storage system is employed, wherein data is encrypted and decrypted and stored through fragmentation and recombination using established techniques, ensuring a secure data management. Statistical features are extracted using a statistical analysis method, and frequency-domain features are extracted using a Fourier transform. A mutual information-based frequency-domain feature selection algorithm is then introduced to identify key frequency components associated with a patient's health status, thereby improving the accuracy and efficiency of data analysis. Statistical features and frequency-domain features are then fused into a comprehensive feature vector using a feature fusion algorithm. A machine learning algorithm is employed to establish a health status prediction model, generate a prediction result of the patient, and produce a health status report of the patient, which can be accessed conveniently by both physicians and patients at any time and from any location. A health data security encryption technique based on the AES (Advanced Encryption Standard) algorithm is employed to encrypt the health status report of a patient, ensuring that only authorized medical personnel and the patient have access to the data. This protects patient privacy and prevents data leakage or unauthorized access. Additionally, a fast and secure transmission method is introduced, using data compression techniques to enable efficient transmission, reduce transmission time, and increase transmission speed, allowing physicians and patients to receive a health report more quickly.

The present disclosure has been described with reference to flowcharts and/or block diagrams of methods, apparatuses (systems), and computer program products according to various embodiments. It should be appreciated that each process and/or block in the flowcharts and/or block diagrams can be implemented by computer program instructions. These computer program instructions may be provided to a processor of a general-purpose computer, special-purpose computer, embedded processor, or other programmable data processing apparatus to produce a machine, such that the instructions, when executed by the processor of the computer or other programmable data processing apparatus, create means for implementing the functions specified in one or more blocks of the flowcharts and/or block diagrams.

These computer program instructions may also be loaded onto a computer or other programmable data processing apparatus to cause a series of operational steps to be performed on the computer or other programmable apparatus, thereby producing a computer-implemented process such that the instructions, when executed, provide steps for implementing the functions specified in one or more blocks of the flowcharts and/or block diagrams.

Although some embodiments of the present disclosure have been described, those skilled in the art, upon learning the basic inventive concepts disclosed herein, may make various changes and modifications to the described embodiments. Accordingly, the appended claims are intended to be interpreted to include preferred embodiments as well as all such changes and modifications that fall within the scope of the present disclosure.

It should be noted that various embodiments of the present disclosure allow for modifications and refinements to be made by those skilled in the art without departing from the principles of the present disclosure. Such modifications and refinements should also be regarded as falling within the scope of the present disclosure.

What is claimed is:

1. A method for monitoring vital signs of a post-operative organ transplant patient, comprising:
collecting patient information, selecting a wearable device for the patient, and monitoring real time vital signs data of the patient using the wearable device, comprehensively harmonizing the real time vital signs data acquired from the wearable device, and storing the vital signs data after comprehensive harmonization;
retrieving the vital signs data of the patient, verifying and correcting the vital signs data of the patient using a data verification and correction algorithm, and processing and analyzing the vital signs data of the patient after verification and correction, so as to generate a health status report of the patient; and
encrypting the health status report of the patient using a health security encryption technology, and introducing a fast and secure transmission method.

2. The method according to claim 1, further comprises:
collecting the patient information from a hospital medical record system;
selecting the wearable device based on the patient information by medical personnel; and
capturing the real time vital signs data of the patient using the wearable device.

3. The method according to claim 2, wherein comprehensively harmonizing the real time vital signs data is performed by introducing:
a data cleansing and purification algorithm for cleansing and purifying the real time vital signs data,
a data denoising algorithm for reducing noise in the real time vital signs, and
a data normalization and balancing algorithm for balancing the real time vital signs data.

4. The method according to claim 3, further comprising:
applying a deep learning-based data compression algorithm to the vital signs data after the comprehensive harmonization, automatically selecting an optimal compression ratio based on characteristics of the vital signs data to compress the vital signs data;
encoding, decoding, and parsing compressed data to form a data packet, thereby obtaining a packaged and compressed patient vital signs dataset;
transmitting and storing the packaged and compressed patient vital signs data; and
introducing a blockchain-based data transmission protocol to transmit the packaged and compressed patient vital signs data to a database for storage.

5. The method according to claim 1, further comprising:
retrieving the vital signs data of the patient from a database;
extracting features from the vital signs data of the patient after verification and correction to obtain a comprehensive feature vector representing the vital signs of the patient; and
analyzing the comprehensive feature vector representing the vital signs of the patient to generate a health status report of the patient.

6. The method according to claim 5, further comprising:
extracting statistical features from the vital signs data of the patient after verification and correction using a statistical analysis method, and calculating statistical characteristics of each vital sign;
transforming the vital signs data of the patient from a time domain to a frequency-domain using a Fourier transform to obtain frequency-domain feature values of the vital signs data; and
introducing a mutual information-based frequency-domain feature selection algorithm to obtain filtered frequency-domain feature values.

7. The method according to claim 6, further comprising:
introducing a comprehensive feature fusion algorithm, wherein the comprehensive feature fusion algorithm fuses the statistical features of the vital signs of the patient and the filtered frequency-domain features to form a comprehensive feature vector.

8. The method according to claim 7, further comprising:
standardizing the comprehensive feature vector of the vital signs of the patient during data analysis;
selecting features most relevant to a health status of the patient using a principal component analysis algorithm;
establishing a health status prediction model under training of a machine learning model;
obtaining a predicted health status result of the patient using the health status prediction model; and
introducing a regularized support vector machine algorithm during training of the health status prediction model.

9. The method according to claim 1, further comprising:
encrypting the health status report of the patient using a health security encryption technology; and
optimizing the health security encryption technology.
